(19) United States
(12) Patent Application Publication
(10) Pub. No.: US 2024/0108316 A1
(43) Pub. Date: Apr. 4, 2024

(54) Processing an Arterial Doppler Ultrasound Waveform

(71) Applicant: Imperial College Innovations Limited, London (GB)

(72) Inventors: Pasha Normahani, London (GB); Usman Jaffer, London (GB)

(73) Assignee: Imperial College Innovations Limited, London (GB)

(21) Appl. No.: 18/275,979

(22) PCT Filed: Jan. 31, 2022
(86) PCT No.: PCT/GB2022/000012

§ 371 Date: Aug. 4, 2023

(30) Foreign Application Priority Data
Feb. 5, 2021 (GB) 2101599.5

(51) Int. Cl. A61B 8/08; A61B 8/00; G06N 20/10

(52) U.S. Cl. CPC A61B 8/5223; A61B 8/0891; A61B 8/488; A61B 8/5269; A61B 8/565; G06N 20/10

(57) ABSTRACT

A computer-implemented method is disclosed. The method comprises classifying an arterial Doppler ultrasound waveform using the arterial Doppler ultrasound waveform and/or a set of features extracted from the arterial Doppler ultrasound waveform using one or more trained machine learning models to identify whether a peripheral arterial disease condition is present and/or to predict a medical outcome related to peripheral arterial disease. The method comprises, upon identifying the presence of the peripheral arterial disease condition and/or predicting the medical outcome, signalling the presence of the peripheral arterial disease condition and/or the medical outcome.

### BACKGROUND

Peripheral arterial disease (PAD) is a major global health problem which is estimated to effect over 230 million people worldwide. It is characterised by progressive atherosclerotic stenosis and occlusion of the lower limb arteries resulting in reduced blood flow and tissue perfusion. Diabetes is an important risk factor for PAD, and the dangerous synergy between the two conditions is associated with poor clinical outcomes, such as increased risk of diabetic foot ulceration, lower limb amputation, myocardial infarction, stroke and mortality.

The diagnosis of PAD in people with diabetes is important and allows for an opportunity to reduce cardiovascular morbidity and mortality through risk factor modification and optimisation of best medical therapy, including the use of antiplatelet and lipid lowering treatment. Furthermore, enhanced ulcer prevention strategies such as frequent foot checks, the provision of orthotic footwear and inserts can be adopted to reduce the risk of ulceration. In patients with active ulceration, the detection of PAD may indicate the need for timely revascularisation to promote healing and reduce the risk of amputation.

The typical clinical presentations of PAD may be absent, subtle or atypical in patients with diabetes who often suffer from accompanying peripheral neuropathy with impaired sensation and are also more likely to suffer from diffuse or distal atherosclerotic disease. Therefore, bedside tests serve an important role in the diagnosis of PAD. Point-of-care duplex ultrasound (DUS) has been shown to be the most accurate bedside test for the detection of PAD in patients with diabetes. It allows for detailed morphological assessment of Doppler arterial spectral waveforms, sampled from the distal posterior tibial and anterior tibial arteries, at the level of the ankle. A haemodynamically significant proximal arterial lesion results in morphological change in the downstream waveform sampled at the level of the ankle. However, identifying pathology through waveforms can be complex as a number of morphological abnormalities can occur, such as loss of pulsatility, long systolic rise time, waveform broadening and long forward flow. Incorporating these adverse features into the definition for pathological waveforms can improve overall diagnostic accuracy. However, a barrier to the implementation of point-of-care DUS in routine clinical practice is the recognition of these adverse waveform features.

Elsie Gyang Ross, Nigam H Shah, Ronald L Dalman, Kevin T Nead, John P Cooke, and Nicholas J Leeper: “The use of machine learning for the identification of peripheral artery disease and future mortality risk”, Journal of Vascular Surgery, volume 64, pp 1512 to 4855 (2016) describes machine learning algorithms for identification of disease and the prognostication of mortality risk. Disparate data inputs are used, and variables collected included demographic variables, clinical comorbidities, medications, lab tests, physical exam variables, physical activity and smoking behaviors, socioeconomic variables, selected genomic markers associated with PAD as well as results of coronary angiograms.

Sooho Kim, Jin-Oh Hahn and Byeng Dong Youn: “Detection and Severity Assessment of Peripheral Occlusive Artery Disease via Deep Learning Analysis of Arterial Pulse Waveforms: Proof-of-Concept and Potential Challenges”, Frontiers in Bioengineering and Biotechnology, volume 8, Article 720 (2020) describes a proof-of concept study involving deep learning-based arterial pulse waveform analysis for detecting and assessing the severity of PAD. Using a transmission line model of arterial haemodynamics, virtual patients associated with PAD of a wide range of severity and the corresponding arterial pulse waveform data were created.

### SUMMARY

According to a first aspect of the present invention there is provided a computer-implemented (or “data processing”) method comprising classifying an arterial Doppler ultrasound waveform using the arterial Doppler ultrasound waveform and/or a set of features extracted from the arterial Doppler ultrasound waveform using one or more trained machine learning models to identify whether a peripheral arterial disease condition is present and/or to predict a medical outcome related to peripheral arterial disease. The method comprises, upon identifying the presence of the peripheral arterial disease condition and/or predicting the medical outcome, signalling the presence of the peripheral arterial disease condition and/or the medical outcome.

The method can help to interpret arterial Doppler ultrasound waveforms consistently, which might otherwise be subject to interobserver variation, and so help to identify whether a peripheral arterial disease condition is present. Additionally or alternatively, the method can help to predict a medical outcome related to peripheral arterial disease. The method can be performed in real-time.

The method may further comprise receiving arterial Doppler ultrasound waveform and extracting the features from arterial Doppler ultrasound waveform.

The arterial Doppler ultrasound waveform is preferably obtained by pulsed-wave Doppler ultrasound.

The features may include a set of time-domain statistical features; and/or a set of time-frequency domain features. The set of time-domain statistical features may include at least one selected from the group consisting of kurtosis, skewness, peak value, mean, standard deviation (STD), root mean square (RMS), impulse factor, crest factor, clearance factor, signal to noise ratio (SNR), total harmonic distortion (THD), signal to noise and distortion ratio (SINAD) and shape factor. The set of time-domain statistical features may include all, substantially all or the majority of the features in the group.

The method may further comprise receiving an image of the arterial Doppler ultrasound waveform and reconstructing the arterial Doppler ultrasound waveform from the image.

The method may further comprise performing signal smoothing of the arterial Doppler ultrasound waveform prior to extracting features.

Classifying the arterial Doppler ultrasound waveform using the arterial Doppler ultrasound waveform using one or more trained machine learning models may comprise using a first machine learning model which is a recurrent neural network (RNN). The RNN may be a long short-term memory (LSTM) network. The arterial Doppler ultrasound waveform preferably comprises a decimated time-varying signal comprising N samples.

Classifying the features extracted from the arterial Doppler ultrasound waveform using one or more trained machine learning models may comprise using a second machine learning model which is based on a supervised machined learning algorithm. The second machine learning model may be based on a support-vector machine (SVM) or logistic regression.

According to a second aspect of the present invention there is provided a computer program which, when executed by at least one processor, performs the method of the first aspect.

According to a third aspect of the present invention there is provided a computer program product comprising a computer-readable medium, which may be non-transitory, storing thereon the computer program of the second aspect.

According to a fourth aspect of the present invention there is provided a machine learning classifier, comprising at least one processor. The at least one processor is configured to classify an arterial Doppler ultrasound waveform using the arterial Doppler ultrasound waveform and/or a set of features extracted from the arterial Doppler ultrasound waveform using one or more trained machine learning models to identify whether a peripheral arterial disease condition is present and/or to predict a medical outcome related to peripheral arterial disease. The at least one processor is configured, upon identifying the presence of the peripheral arterial disease condition and/or predicting the medical outcome, to signal the presence of the peripheral arterial disease condition and/or the medical outcome.

The at least one processor may comprise at least one central processing unit (CPU). The at least one processor may comprise a graphical processing unit (GPU).

The machine learning classifier may further comprise memory and/or storage for storing the one or more trained machine learning models.

The at least one processor may be configured to receive the arterial Doppler ultrasound waveform and to extract the features from the arterial Doppler ultrasound waveform.

According to a fifth aspect of the present invention there is provided a medical ultrasound scanner comprising an ultrasound transceiver for generating an arterial Doppler ultrasound waveform, an optional signal processor, and the machine learning classifier of the fourth aspect. The ultrasound transceiver is configured to provide the arterial Doppler ultrasound waveform to the signal processor and/or to the system and the signal processor is configured to extract features from the arterial Doppler ultrasound waveform and to provide the features to the system.

The medical ultrasound scanner may be a portable ultrasound machine. The medical ultrasound scanner may be a point-of-care ultrasound machine. The medical ultrasound scanner may be configured for duplex ultrasound. The medical ultrasound scanner preferably is operable in pulsed-wave Doppler ultrasound mode.

The medical ultrasound scanner may comprise a probe and a base unit. The probe may be linked to the base unit by a wired link or by a wireless link, such as a BlueTooth® link.

According to a sixth aspect of the present invention there is provided a medical ultrasound scanner having a communications network interface and a server having a communications interface, the server comprising the machine learning classifier of the fourth aspect. The medical ultrasound scanner is configured to transmit the arterial Doppler ultrasound waveform and/or a set of features to the server and the server is configured to identify the presence of the peripheral arterial disease condition and/or the medical outcome to the medical ultrasound scanner or another system or device. The other system or device may be a computer system, such as a tablet computer or smart phone, or a medical records database.

According to a seventh aspect of the present invention there is provided a computer-implemented method, comprising training one or more machine learning trainers using a plurality of arterial Doppler ultrasound waveforms as a training set and/or a plurality of sets of features extracted from respective arterial Doppler ultrasound waveforms as a training set, wherein each one of the plurality of arterial Doppler ultrasound waveforms and each one of the sets of features are labelled as to the presence of a peripheral arterial disease condition and/or a prediction of a medical outcome related to peripheral arterial disease, and storing one or more trained machine learning models obtained from training the one or more machine learning trainers.

The one or more machine learning trainers may include a first machine learning trainer is based on a recurrent neural network and/or a second machine learning trainer is based on a supervised machine learning algorithm, such as a support-vector machine or logistic regression.

According to an eight aspect of the present invention there is provided a computer program which, when executed by at least one processor, performs the method of the seventh aspect.

According to a ninth aspect of the present invention there is provided a computer product comprising a computer-readable medium, which may be non-transitory, storing thereon the computer program of the eighth aspect.

According to a tenth aspect of the present invention there is provided a machine learning trainer, comprising at least one processor and storage. The at least one processor is configured to train one or more machine learning trainers using a plurality of arterial Doppler ultrasound waveforms as a training set and/or a plurality of sets of features extracted from respective arterial Doppler ultrasound waveforms as a training set, wherein each one of the plurality of arterial Doppler ultrasound waveforms and each one of the sets of features are labelled as to the presence of a peripheral arterial disease condition and/or a prediction of a medical outcome related to peripheral arterial disease, and to store one or more trained machine learning models from the one or more machine learning trainers.

The at least one processor may comprise at least one central processing unit (CPU). The at least one processor may comprise a graphical processing unit (GPU).

### BRIEF DESCRIPTION OF THE DRAWINGS

Certain embodiments of the present invention will now be described, by way of example, with reference to the accompanying drawings, in which:

FIG. 1 illustrates ultrasonography of distal anterior and posterior tibial arteries in an ankle of a patient using a medical ultrasound scanner;

FIG. 2 is a schematic block diagram of a computer system;

FIG. 3 is a process flow diagram of processing an image of an arterial spectral waveform obtained by a medical ultrasound scanner;

FIG. 4A is an example of an image of a captured Doppler arterial spectral waveform;

FIG. 4B is an example of a waveform after signal reconstruction and pre-processing;

FIG. 5 illustrates N-level decomposition of a Doppler arterial spectral waveform;

FIG. 6 illustrates an example of Doppler arterial spectral waveform;

FIG. 7 illustrates the first five levels of approximation coefficients obtained by deconstructing the waveform shown in FIG. 6 using discrete wavelet transform (DWT);

FIG. 8 illustrates the first five levels of detail coefficients obtained by deconstructing the waveform shown in FIG. 6 using DWT;

FIG. 9 is a block diagram of a long short-term memory (LSTM) network;

FIG. 10 schematically illustrates training of the LSTM network shown in FIG. 9;

FIG. 11 illustrates training a logistic regression classifier;

FIG. 12 illustrates training a support vector machine (SVM);

FIG. 13 is an area under receiver operating characteristics curve (AURUOC) for a logistic regression model using a combination of time-domain statistical and time-frequency domain multiscale wavelet variance features, wherein x marks current classifier performance on the ROC curve;

FIG. 14 is a schematic block diagram of a medical ultrasound scanner which includes classifier for classifying an arterial spectral waveform obtained by the medical ultrasound scanner;

FIG. 15 illustrates classification of an arterial spectral waveform;

FIG. 16 is a schematic block diagram of a medical ultrasound machine and a remote server having a classifier for remotely classifying an arterial spectral waveform obtained by the medical ultrasound scanner; and

FIG. 17 is a schematic block diagram of remote server which can receive a plurality of arterial spectral waveforms and generate a trained model.

### DETAILED DESCRIPTION OF CERTAIN EMBODIMENTS

Introduction

Machine learning techniques allow for non-linear classification of hard-to-define physiological signals. This approach can help to reduce inter-observer variation and facilitate adoption of point-of-care DUS for the detection of PAD in diabetes.

As will be described hereinafter, a study was conducted to evaluate the utility of machine learning techniques for the diagnosis of PAD from Doppler arterial spectral waveforms sampled at the level of the ankle in patients with diabetes. Application of a long short-term memory network (LSTM) for the classification of raw signals, as well as logistic regression and support vector machine classification using extracted time- and time-frequency domain features, are described.

Dataset

This study is based on a prospectively recruited cohort of 305 patients with diabetes who presented to two diabetic foot clinics in London, United Kingdom (TEsting for Arterial Disease in Diabetes study, TrEAD). Reference is made to P. Normahani, S Poushpas, M Alaa, V Bravis, M Aslam, U Jaffer: “Diagnostic accuracy of point-of-care tests used to detect arterial disease in diabetes: TEsting for Arterial disease in Diabetes (TrEAD) study”, Annals of Surgery, 2021 (herein after referred to as “P. Normahani et al. Reference 1”) and P. Normahani, S Poushpas, M Alaa, V Bravis, M Aslam, U Jaffer: “Study protocol for a comparative diagnostic accuracy study of bedside tests used to detect arterial disease in diabetes: TEsting for Arterial disease in Diabetes (TrEAD) study”, BMJ Open. 2020 Feb. 5; 10(2):e033753 (herein after referred to as “P. Normahani et al. Reference 2”).

The TrEAD study aimed to evaluate the diagnostic accuracy of point-of-care DUS and other commonly used bedside tests for the detection of PAD in patients with diabetes as compared to a blinded reference test of a full lower limb DUS. The study was approved by the Health Research Authority (REC reference 17/LO/1447). Every patient gave written informed consent to take part in the study. The TrEAD protocol and details of patient recruitment and data acquisition are described in P. Normahani Reference 1 ibid. and P. Normahani Reference 2 ibid.

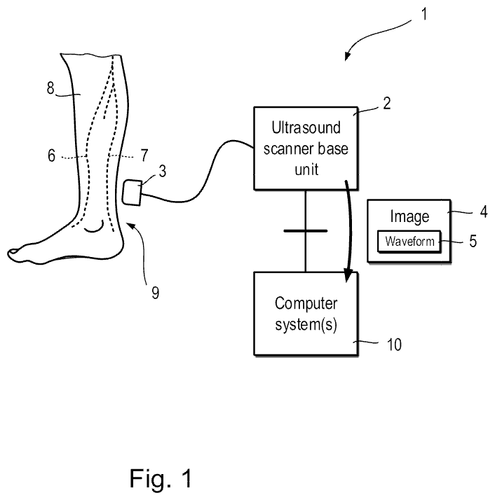

Referring to FIG. 1, point-of-care DUS was performed using an ultrasound machine 1 (herein also referred to as a “medical ultrasound scanner” or “medical ultrasound system”), in particular a portable ultrasound machine 1 in the form of a Mindray M7 (Shenzhen, China) with a linear 6-14 MHz transducer by a vascular scientist. The ultrasound machine 1 has a base unit 2 and a probe 3 connected by a wired link. However, a wireless (e.g., BlueTooth®) link may be used. Images 4 of all arterial spectral waveforms 5 sampled at the distal anterior and posterior tibial arteries 6, 7 in the lower limb 8 at the level of the ankle 9 were collected. Blinded full lower limb reference DUS results were used to label each arterial spectral waveform according to PAD status (i.e., PAD, no-PAD). PAD was defined as the presence of occlusions, or stenosis, or diffuse stenotic disease, which individually or collectively, caused significant velocity change (PSVR 22 represents a 50% stenosis) and flow disturbance locally, and resulted in biphasic or monophasic signal distally.

Pulsed-wave Doppler ultrasound is used. However, the approach herein described can used with measurements collected using continuous-wave Doppler ultrasound.

As will be explained in more detail, the image 4 or waveform 5 (or “signal”) are processed using one or more computer systems 10.

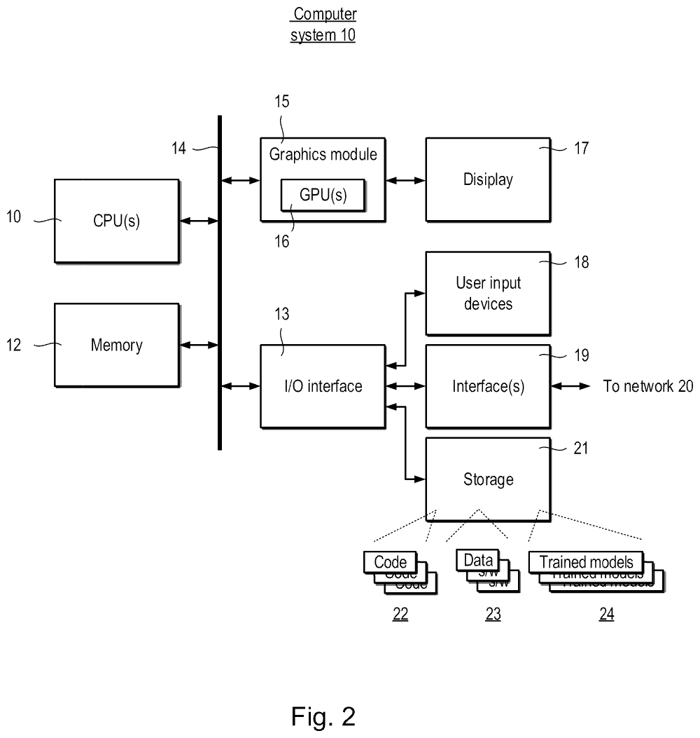

Referring to FIG. 2, a suitable computer system 10 for processing images 4 and/or ultrasound signals 5 comprises at least one central processing unit (CPU) 11 (or “processor”) 11, memory 12 and an input/output interface 13 interconnected by a bus system 14. The system 10 may include a graphics module 15, which includes a graphical processing unit (“GPU”) 16, and a display 17. The system 10 may include user input device(s) 18 such as keyboard (not shown) and pointing device (not show). The system 10 includes network interface(s) 19 to communications network 20 and storage 21 for example in the form of hard-disk drive(s) and/or solid-state drive. The storage 21 may store one or more sets of code 22 (which may also be referred to as “instructions”, a “program” or “software”) for reconstructing, resampling and/or smoothing signals, for extracting features and/or for training a machine learning network or model. One or more sets of code 22 may take the form of a function or sub-routine in a software package or programming environment. The storage 21 may store one or more sets of data 23 which may include different types of data, for example, signals and extracted features, and parameters or options, and/or trained models (or “trained networks”) 24.

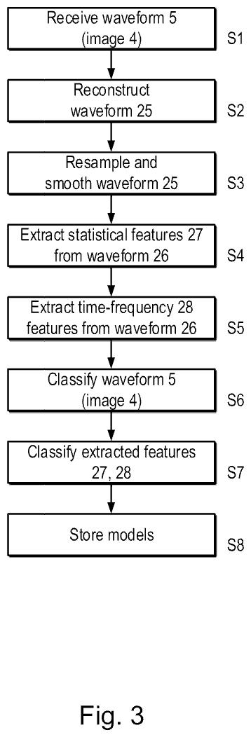

Referring to FIG. 3, a method of processing an image 5 of an arterial spectral waveform 4 (steps S1 to S8) obtained by a medical ultrasound scanner 1 is shown.

Signal Reconstruction

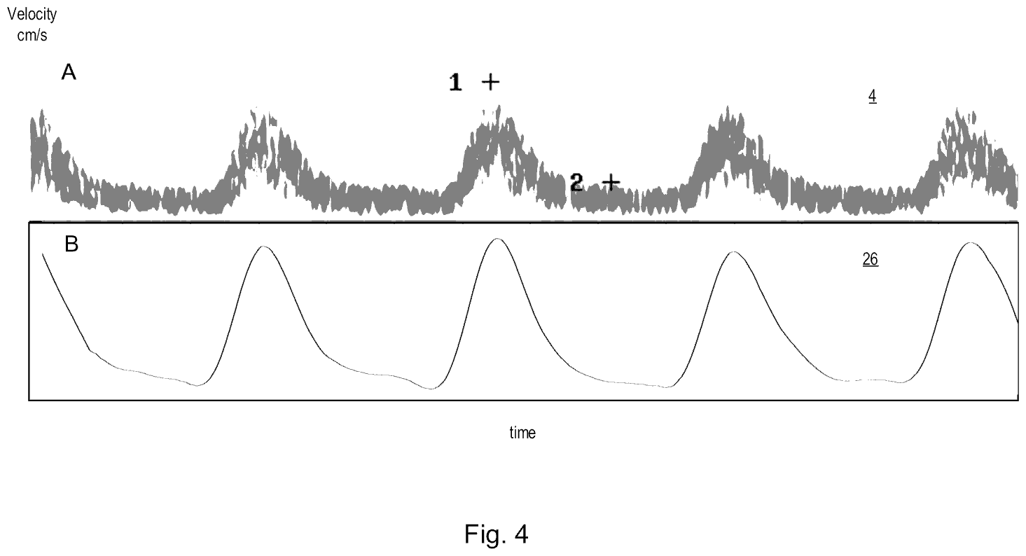

Referring also to FIG. 3 and FIGS. 4A and 4B, all waveform images 4 were uploaded to R (version 3.3.1; R Foundation for Statistical Computing, Vienna, Austria) and the signals 5 are individually reconstructed using the R package Digitize (version 0.0.4) (step S2). First, the x (time, second) and y axes (peak systolic velocity, cm/s) for each image 4. Then, the outer envelope of the waveform was manually demarcated. This process generates calibrated x, y coordinates for each signal 5.

R need not be used and signal reconstruction can be performed locally using a computer system 10 (FIG. 2). If the image 4 is a colour image, then it is converted into to a grey scale image (step SR1). Unwanted labels/elements (which can be identified by pixel intensity) are removed from the image (step SR2). The y-coordinate of the baseline is identified (step SR3). Identification of the baseline is used to identify negative and positive velocity values. The baseline (which can be identified based on pixel intensity) is removed from the image (step SR4). The edge of the waveform is detected (for example, using a MATLAB function), then the edge is dilated, filled and smoothed to leave the outer edge remaining (step SR5). The coordinates of waveform edge are extracted (step SR6). As the y coordinate of the baseline is already known, inflection points above and below the baseline can be adjusted to give x-y values for the waveform edge (step SR7). Since the x-y coordinates are arbitrary, they are rescaled to reflect true values of velocity and time (step SR8). The y-coordinate values can be rescaled based on user-defined peak systolic velocity. The x-coordinate values can be rescaled based on user-defined total sampling time.

Reconstructed signals 25 were exported to MATLAB (version R2020b; The Mathworks Inc., Natick, Massachusetts, USA), resampled using nearest neighbour method at a predefined time step of 0.0001 seconds and synchronised (step S3). Each image captured 3.5 seconds of signal recording. To account for late starts in recording all signals 25 were extracted from 0.5 to 3.5 seconds and the resulting signals were 3 seconds long (i.e., 30000 samples). Signal smoothing was performed using the Savitzky-Golay method. An example of a reconstructed and smoothed signal 26 is shown in FIG. 4B.

Extraction and Selection of Statistical and Time-Frequency Domain Features

To reduce the dimension of the data, time and time-frequency domain features 27, 28 of potential importance were extracted (step S4 & S).

Thirteen time-domain statistical features 27 were extracted, namely kurtosis, skewness, peak value, mean, standard deviation (STD), root mean square (RMS), impulse factor, crest factor, clearance factor, signal to noise ratio (SNR), total harmonic distortion (THD), signal to noise and distortion ratio (SINAD) and shape factor.

Definitions for these features 27 are given in Table S1 below:

TABLE S1

| Feature | Definition |
|---|---|
| Mean | Mean value of the signal |
| Standard deviation | Standard deviation of the signal |
| (STD) | |
| Peak value | Maximum absolute value of the signal |
| Root mean square | Square root of the average of the squared values |
| (RMS) | of the waveform |
| Skewness | The degree of symmetry of distribution around |
| | signal mean |
| Kurtosis | Distribution of observed data around the mean, |
| | i.e., the length of the tails of a signal distribution |
| Impulse factor | Heigh of the peak value as compared to the mean |
| | level of the signal |
| Crest factor | Ratio of peak value over RMS and indicates the |
| | shape of the waveform |
| Shape factor | RMS divided by the mean of the absolute value |
| Clearance factor | Ratio of peak value over the squared mean value of |
| | the square roots of the absolute amplitudes |
| Signal to noise ratio | Ratio of signal power to noise power |
| (SNR) | |
| Total harmonic | Ratio of total harmonic power to fundamental |
| distortion (THD) | power |
| Signal to noise and | Ratio of total signal power to total noise-plus- |
| distortion ratio | distortion power |
| (SINAD) | |

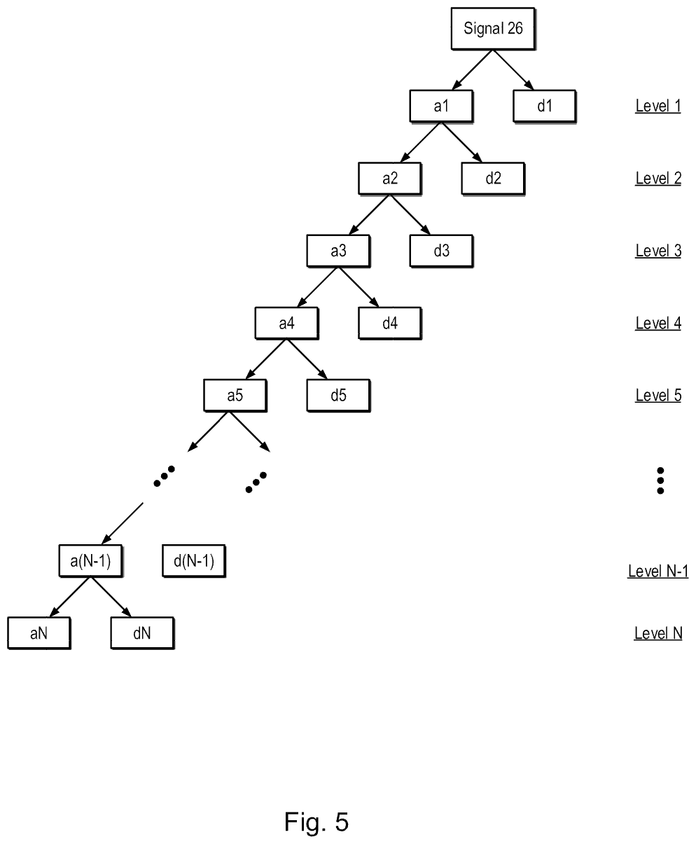

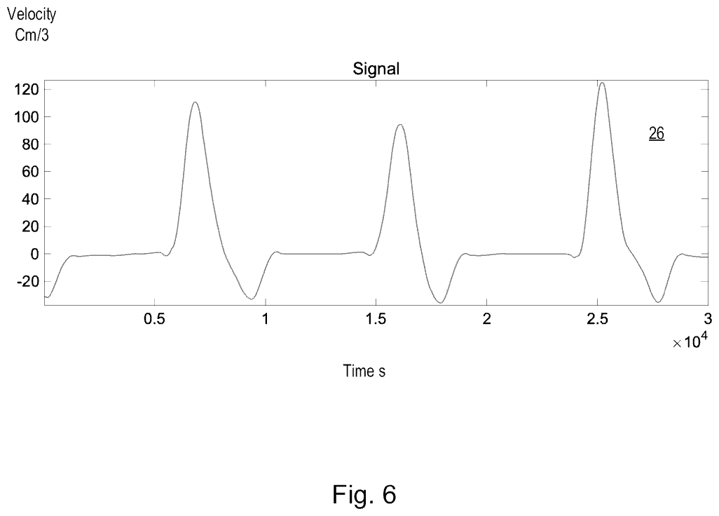

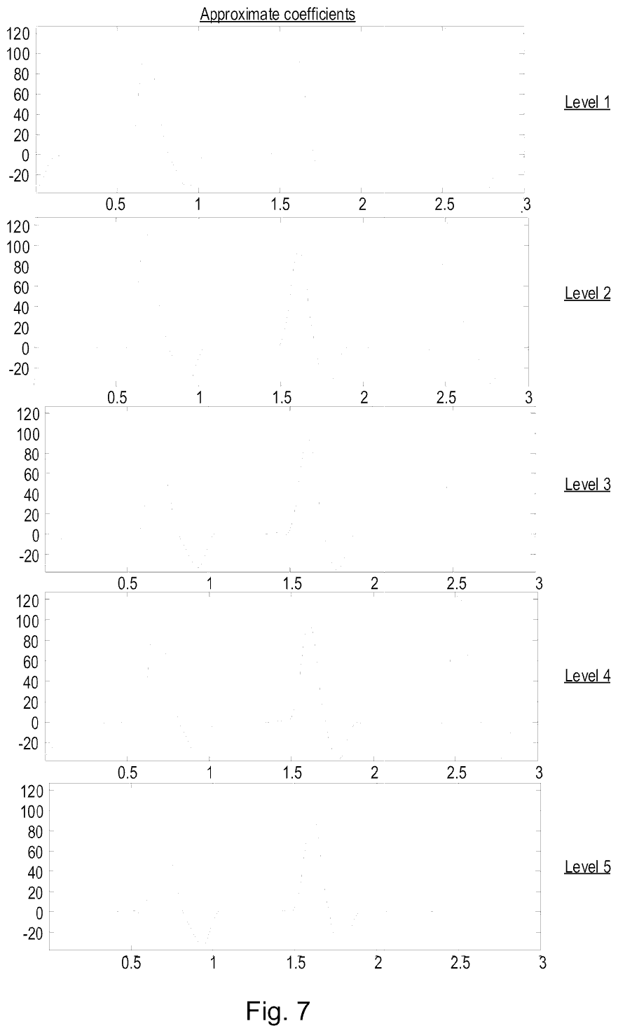

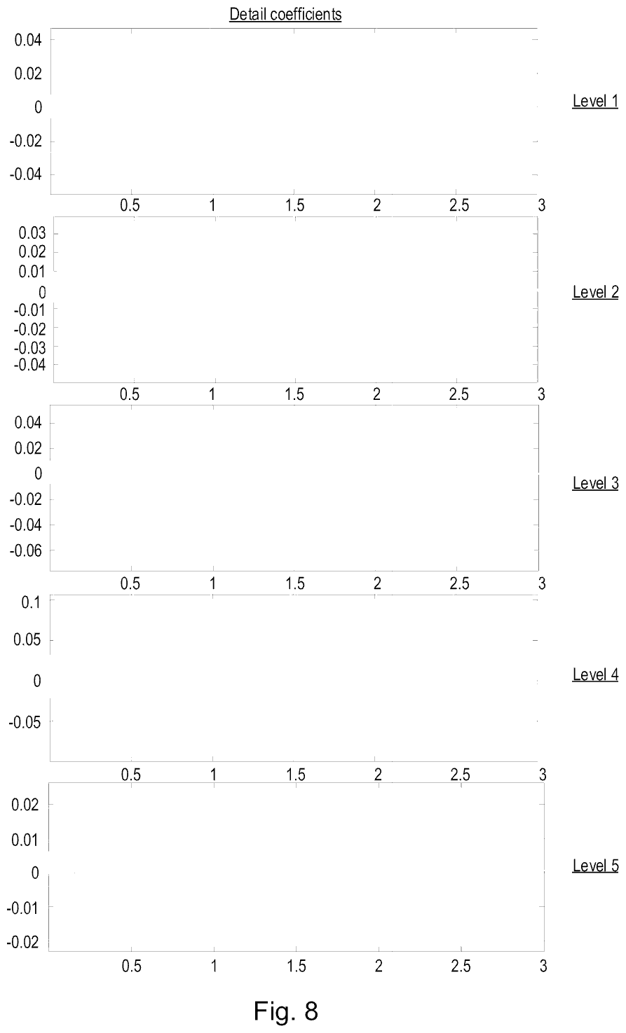

Signal to noise and distortion Ratio of total signal power to total noise-plus-ratio (SINAD) distortion power Referring to FIGS. 5, 6, 7 and 8, time-frequency domain features 28 were extracted using discrete wavelet transform (DWT) which deconstructs a signal 26 into frequency sub-bands (or “scales”). DWT captures and localises transient features in time series data. By decomposing a signal 26 into components of different scales, DWT allows for the detection of variations across scales in observed data. Multiscale wavelet variance estimates were extracted from each signal over the entire data length. Multiscale wavelet variance estimates can be used to distinguishing between different ECG signals and reference is made to E. Maharaj and A. Alonso: “Discriminant analysis of multivariate time series: Application to diagnosis based on ECG signals”, Computational Statistics & Data Analysis, volume 70, pages 67 to 87 (2014). Wavelet filter of length 2 of the Daubechies family (db2) was used to generate DWT coefficients and, hence, the DWT variance of the signal. The number of scales was set at 13, resulting in 14 possible features, i.e., 13 detail coefficients and 1 approximation coefficient.

Differences in the 27 possible features were compared between the PAD and no-PAD groups using independent T-test and only those with a statistically significant difference were retained.

Machine Learning

A long short-term memory (LSTM) network was used for the classification of raw signals, i.e., images 4, and logistic regression and support vector machine (SVM) were used for classification of extracted features 27, 28 (step S6 & S7). Data are structured as a table using numerical features and categorical labels. Signals and feature vectors were randomly divided into training (80%) and testing (20%) sets. All models were trained using a CPU 10 (FIG. 2).

Long Short-Term Memory Network Classification of Raw Signals

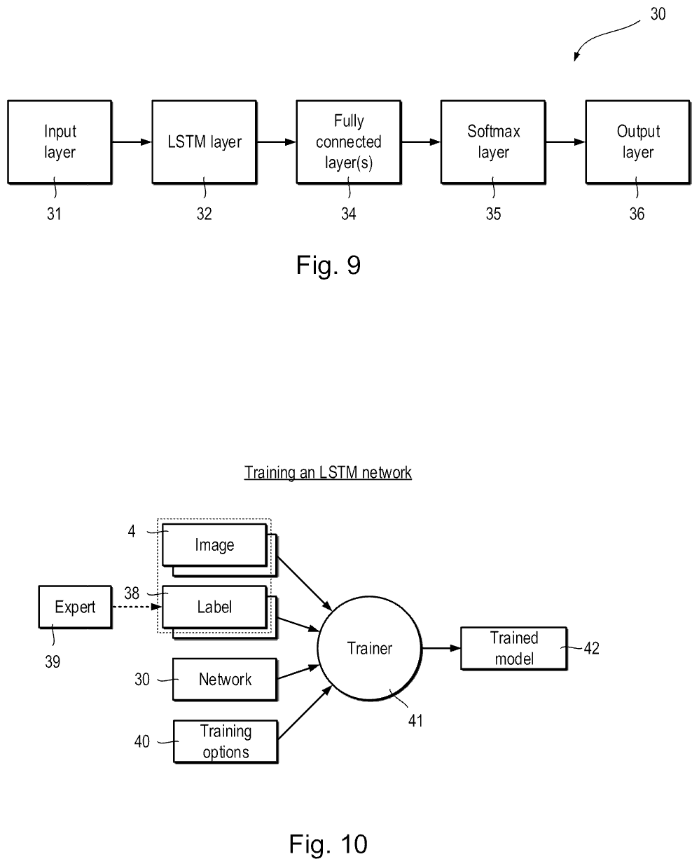

Referring to FIG. 9, an LSTM network 30 was used for classification of raw signals, i.e., images 4.

The LSTM network 30 includes an input layer 31, an LSTM layer 32 having 100 hidden units 33, a fully connected layer 34 of size 2, a softmax layer 35 and an output layer 36.

Referring to FIG. 10, images 4, corresponding labels 38 which are provided by an expert 39, and the LSTM network 30, together with training options 40, are supplied to a trainer (or “builder” or “learner”) 41 to create a trained LSTM network (or “trained model”) 42.

In this case, a single-input, bi-directional LSTM network 30 was trained using binary cross-entropy loss for a maximum of 10 epochs on mini-batches of size 15, initial learning rate of 0.01 and sequence length of 15000. The network 30 included of two fully connected layers. To achieve the same number of signals in each class (i.e., PAD and no-PAD) oversampling was performed.

In particular, the LSTM network 30 is created and trained using MATLAB® (version R2020b; The Mathworks Inc., Natick, Massachusetts, USA). The function layer is used to create an LSTM layer in an array Layers and the trainNetwork function is used to train the LSTM network and create a trained LSTM network net. The LSTM network 30 can be created and trained using other packages, such as Keras.

Logistic Regression and Support Vector Machine Classification of Extracted Features

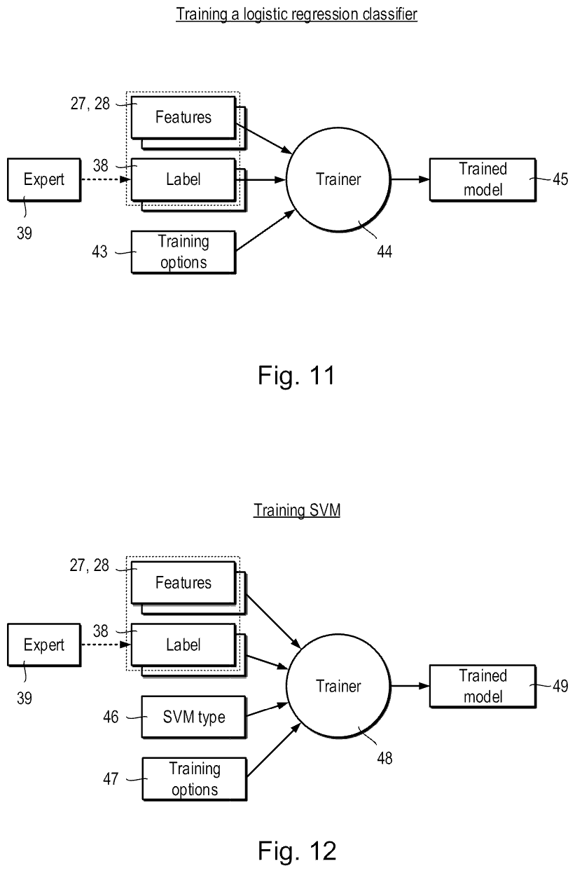

Referring to FIG. 11, features 27, 28 and corresponding labels 38, and training options 43, are supplied to a trainer (or “builder” or “learner”) 44 to create a trained logistic regression classifier (or “trained model”) 45.

Referring to FIG. 12, features 27, 28 and corresponding labels 38 which are provided by an expert 39, SVM type 46 and training options 47, are supplied to a trainer (or “builder” or “learner”) 48 to create a trained SVM classifier (or “trained model”) 45.

In this case, the fitglm and fitcsvm functions in MATLAB (version R2020b; The Mathworks Inc., Natick, Massachusetts, USA) are used for logistic regression and support vector machine leaning respectively.

In particular, a linear kernel SVM is used with Kernel scale set to “Automatic”, box constraint level, multiclass method 1v1 and standardised data set to “True”.

As will be shown in more detail later, the linear kernel SVM and logistic regression models were compared using three sets of features, namely all combined features 27, 28, only the multiscale wavelet variance features 27, and only the statistical features 28.

Other forms of supervised machine learning can be used, such as a convolutional neural network (CNN) or other form of artificial neural network, or methods such as Naïve Bayes, decision or classification tree, or K nearest neighbour

The waveform 5 and/or the extracted features 27, 28 can be classified (steps S6 & S7). In other words, both forms of classification need not be performed. Moreover, time-domain statistical features 27 and/or time-frequency domain features 28 can be classified. In other words, both types of features 27, 28 need not be used. However, both can be employed, which can lead to better results.

Results

After excluding occluded vessels which were not amenable to signal extraction, 590 arterial spectral waveforms from 305 patients were available for analysis (PAD 369, no-PAD 221). Baseline demographic data for the 305 study participants are presented in Table 1 below. Stratified demographic data is also included in Table 1 below.

TABLE 1

| | All | | No PAD | | PAD | | p* |
|---|---|---|---|---|---|---|---|
| N | 305 | (%) | 103 | | 202 | | |
| Gender (%) | | | | | | | |
| M | 206 | (67.5) | 61 | | 145 | | 0.037 |
| F | 99 | (32.5) | | | | | |
| Age (median (CI)) | 72 | (62-79) | 66.77 | (15.43) | 71.70 | (12.18) | 0.002 |
| Diabetes mellitus | | | | | | | |
| type (%) | | | | | | | |
| Type 1 | 24 | (7.9) | — | | — | | — |
| Type 2 | 281 | (92.1) | 93 | (90.3) | 188 | (93.1) | 0.53 |
| Diabetes mellitus | 17.15 | (12.52) | 15.42 | (10.28) | 18.02 | (13.46) | 0.087 |
| duration (mean (SD)) | | | | | | | |
| Smoking (%) | | | | | | | |
| Current | 39 | (12.8) | 18 | (17.5) | 21 | (10.4) | |
| Ex- smoker | 126 | (41.3) | 34 | (33.0) | 92 | (45.5) | |
| Non-smoker | 140 | (45.9) | 51 | (49.5) | 89 | (44.1) | |
| Ischaemic heart | | | | | | | |
| disease (%) | | | | | | | |
| Yes | 64 | (21.0) | 17 | (16.5) | 47 | (23.3) | 0.221 |
| No | 241 | (79.0) | | | | | |
| Retinopathy (%) | | | | | | | |
| Yes | 105 | (34.4) | 30 | (29.1) | 75 | (37.1) | 0.206 |
| No | 200 | (65.6) | | | | | |
| Chronic kidney | | | | | | | |
| disease (%) | | | | | | | |
| Yes | 52 | (17.0) | 11 | (10.7) | 41 | (20.3) | 0.051 |
| No | 253 | (83.0) | | | | | |
| Stroke (%) | | | | | | | |
| Yes | 55 | (18.0) | | | | | |
| No | 250 | (82.0) | | | | | |
| Heart failure (%) | | | | | | | |
| Yes | 26 | (8.5) | 3 | (2.9) | 23 | (11.4) | 0.022 |
| No | 279 | (91.5) | | | | | |
| Minor amputation | | | | | | | |
| history (%) | | | | | | | |
| Yes | 37 | (12.1) | 1 | (1.0) | 36 | (17.8) | <0.001 |
| No | 268 | (87.9) | | | | | |
| Major amputation | | | | | | | |
| history (%) | | | | | | | |
| Yes | 0 | (0) | | | | | |
| No | 305 | (100.0) | 103 | (100) | 202 | (100) | NA |
| Neuropathy (%) | | | | | | | |
| Yes | 203 | (66.6) | 54 | (52.4) | 149 | (73.8) | <0.001 |
| No | 102 | (33.4) | | | | | |
| Active ulcer (%) | | | | | | | |
| Yes | 123 | (40.3) | 24 | (23.3) | 99 | (49) | <0.001 |
| No | 182 | (59.7) | | | | | |

Of the 27 possible features, 26 features (13 statistical time-domain and 13 time-frequency multiscale variance features 27, 28) were statistically different between the two groups (see table S2 below) and were used as features for classification and p values are set out in Table S2 below:

TABLE S2

p value

Statistical metrics

| | |
|---|---|
| SNR | 8.41E−185 |
| THD | 2.47E−183 |
| Mean | 2.44E−168 |
| peak value | 6.04E−168 |
| RMS | 2.73E−145 |
| STD | 2.45E−119 |
| Skewness | 2.61E−99 |
| Shape factor | 3.50E−99 |
| SINAD | 3.66E−65 |
| Clearance factor | 7.48E−43 |
| Crest factor | 2.31E−17 |
| Impulse factor | 6.57E−08 |
| Kurtosis | 1.97E−02 |

Multiscale wavelet variance

| | |
|---|---|
| d1 | 7.49E−188 |
| d2 | 7.49E−188 |
| d3 | 7.49E−188 |
| d4 | 7.53E−188 |
| d5 | 7.91E−188 |
| d6 | 1.14E−187 |
| d7 | 8.47E−186 |
| d8 | 2.40E−156 |
| d9 | 0.3463 |
| d10 | 6.96E−41 |
| d11 | 1.15E−49 |
| d12 | 7.87E−39 |
| d13 | 4.58E−28 |
| a13 | 6.48E−06 |

Overall test accuracy, sensitivity and specificity for each classification approach is presented in Table 2 below:

TABLE 2

| | | Acc. | Sens. | Spec. |
|---|---|---|---|---|
| Method | Input | % | % | % |
| LSTM | Raw signal | 78 | 62 | 95 |
| SVM | Combined features | 86 | 90 | 80 |
| SVM | WT features | 82 | 95 | 69 |
| SVM | Statistical features | 86 | 88 | 82 |
| Logistic regression | Combined features | 88 | 92 | 82 |
| Logistic regression | WT features | 80 | 92 | 59 |
| Logistic regression | Statistical features | 83 | 88 | 75 |

Higher overall accuracy was achieved using SVM and logistic regression methods combined with extracted features as compared to LSTM network classification of raw signals. The highest overall accuracy was achieved using both statistical time-domain and time-frequency domain multiscale wavelet variance features in combination with a logistic regression model: 88% accuracy, 92% sensitivity and 82% specificity. The confusion matrix is presented in Table 3.

TABLE 3

| | | PAD | No PAD |
|---|---|---|---|
| True class | PAD | 67 | 6 |
| | No PAD | 8 | 36 |

Predicted class

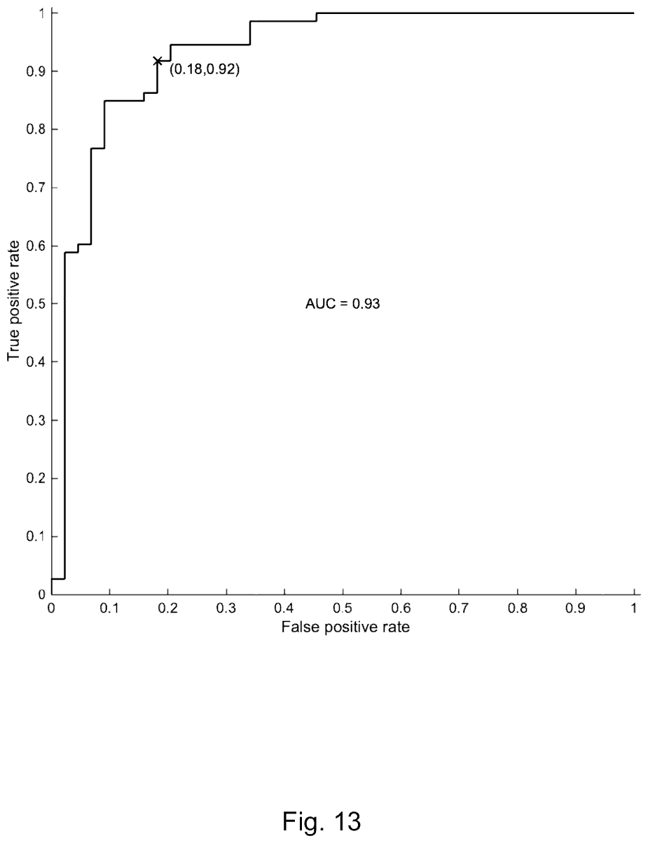

Referring to FIG. 13, the area under receiver operating characteristics curve (AURUOC) metrics show excellent discrimination between PAD and no-PAD groups: AUROC 0.93.

Classification of PAD Severity

Instead of only two categories, namely no PAD and PAD, the data can be split into (i.e., labelled using) three categories, namely, no PAD, mild PAD (50-75% stenosis as determine by reference standard) and moderate/severe PAD (>75% stenosis).

A quadratic SVM was used to classify the 26 extracted features 27, 28. Other parameters used in SVM remained the same and the same 80:20 ratio for training to testing ratio was used.

The confusion matrix for the three-class approach is presented in Table 4 below:

TABLE 4

| | | | | Moderate/ |
|---|---|---|---|---|
| | | No PAD | Mild PAD | severe PAD |
| True | No PAD | 38 | 6 | 0 |
| class | Mild PAD | 4 | 28 | 10 |
| | Moderate/ | 1 | 11 | 20 |
| | severe PAD | | | |

Predicted class

The overall accuracy 72.9%. Although the accuracy is lower than the two-class classification, this is due to the size of the dataset.

Discussion

Strandness et al. reported on morphological differences in Doppler arterial waveforms of normal and atherosclerotic peripheral arteries and reference is made to D. Strandness, E. McCutcheon, R. Rushmer: “Application of a transcutaneous Doppler flowmeter in evaluation of occlusive arterial disease”, Surg. Gynecol. Obstet., vol. 122, no. 5, pp. 1039-45 (1966) and D. Strandness, R. Schultz, D. Sumner and R. Rushmer: “Ultrasonic flow detection. A useful technic in the evaluation of peripheral vascular disease”, Am. J. Surg., vol. 113, no. 3, pp 311-20 (1967). This was followed by rapid technological and methodological advancement which established vascular ultrasound as the single most important non-invasive vascular diagnostic imaging modality. Despite important early efforts to define certain aspects of waveform morphology, such as resistance and pulsatility, in quantitative terms, waveform assessment has not changed for almost 50 years. Reference is made to R. Gosling and D. King: “Arterial assessment by Doppler-shift ultrasound”, Proc. R. Soc. Med., vol. 67, no. 6 (pt. 1), pp. 447-9 (1974) and L. Pourcelot: “Clinical applications of transcutaneous Doppler examinations”, Velocim Ultrason Doppler, ed. P. Peronneau pp. 213-240 (1975).

As herein described, machine learning is applied to the classification of Doppler arterial spectral waveforms for the diagnosis of PAD. It is shown that machine learning can achieve high diagnostic accuracy for PAD from the interpretation of ankle Doppler arterial waveforms. The performance of machine learning (sensitivity 92%, specificity 82%) in this study is comparable to that of waveform interpretation by expert vascular scientists reported in the TrEAD study (sensitivity 95%, specificity 77%) as described in P. Normahani et al. Reference 1 ibid. It can have the added advantage of standardising assessment and helping to eliminate interobserver variation, which represents a significant challenge given the qualitative nature of waveform interpretation. This approach may also shorten the learning curve for point-of-care DUS by removing waveform interpretation as a barrier and hence further facilitate its adoption in routine clinical practice. This can be important given that point-of-care DUS is a bedside test for use by frontline health care professionals looking after patients with diabetes (such as surgeons, podiatrists, nurses and physicians), who are unlikely to have had formal training in vascular ultrasound.

Although the study herein described is related to the specific application of point-of-care DUS for the detection of PAD in patients with diabetes, the findings may have wider implications for the field of vascular ultrasound. Recently, acknowledging significant heterogeneity in waveform interpretation, there has been an attempt to standardise key definitions and descriptors of waveform morphology by expert consensus. However, it remains the case that waveform morphology is complex and is dependent on location in the arterial tree as well as severity of disease. Waveform interpretation will therefore likely remain challenging and will continue to be associated with significant inter-observer variation. Machine learning tools, such as those tested in this study, may be useful in standardising assessment and reducing inter-observer variation when applied to other vessels or patient groups, e.g., carotid imaging.

In this study, higher classification accuracy was found when using statistical time-domain and multiscale wavelet variance time-frequency features 27, 28 for classification, as compared to the raw signal. This, however, may not be an exhaustive list of putative features. Improvements in accuracy might also be achieved by establishing a large repository of waveforms and thereby increasing the size of the dataset available for training, provided these are labelled accurately using a suitable reference test. In this study, a full lower limb DUS was used as a reference standard. This has the advantage of being inexpensive, non-invasive and has also been shown to have a good agreement with intra-arterial digital subtraction angiography (DSA). However, it may be less reliable in interrogating the commonly affected distal vessels in diabetes and may fail to detect isolated atherosclerotic PAD lesions in the foot vessels. Doppler arterial spectral waveform machine learning analysis may be able to detect isolated disease in the foot which would be associated with increased vascular resistance and a change in waveform morphology. It is possible that cases of isolated PAD in the foot may have been present but mislabeled using a chosen reference test. Alternative strategies such as magnetic resonance angiography (MRA) and computed tomography angiography (CTA) may be suitable reference test alternatives.

As herein described, machine learning algorithms have been constructed with high discriminatory ability for the diagnosis of peripheral arterial disease using Doppler arterial spectral waveforms sampled at the ankle vessels.

Prognosis

The approach herein described involves determining the presence or absence of peripheral arterial disease and, optionally, the severity of peripheral arterial disease which can be used in diagnosing the condition. Training involves labelling an arterial Doppler ultrasound waveform knowing whether the condition is present or not and, optionally, if the condition is present, its severity. Thus, a trained machine learning model can be used to classify an unseen arterial Doppler ultrasound waveform to determine whether the condition is present or not and, optionally, its severity.

The approach, however, can be used in predicting medical outcomes, i.e., prognosis. For example, a patient may present with a foot ulcer. In the next six to twelve months, possible medical outcomes include the ulcer healing, the need to amputate the foot or leg, and/or the occurrence of a non-fatal cardiovascular event or the occurrence of a fatal cardiovascular event. Similarly, a patient may present without an ulcer and possible outcomes may include the patient developing an ulcer, the need to amputate, and or the occurrence of a non-fatal or fatal cardiovascular event. Training involves acquiring an arterial Doppler ultrasound waveform, waiting a period of time, for example, six to twelve months, identifying the actual medical outcome, and labelling the earlier-acquired arterial Doppler ultrasound waveform with prognostic labels, such as amputation or cardiovascular event. Thus, machine learning models can be trained and used to classify an unseen arterial Doppler ultrasound waveform of a patient with an ulcer to predict medical outcome(s). Accordingly, suitable medical intervention or preventative measures can be taken such as prescribing medication and changes in lifestyle. Reference is made to P. Normahani et al: “Arterial spectral waveform analysis in the predication or diabetic foot ulcer healing”, Perfusion, (2020) (https://journals.sagepub.com/doi/full/10.1177/0267659120957849).

Examples of arrangements in which a trained model can be used for inferring the presence of PAD or predicting medical outcomes will now be described.

Inference

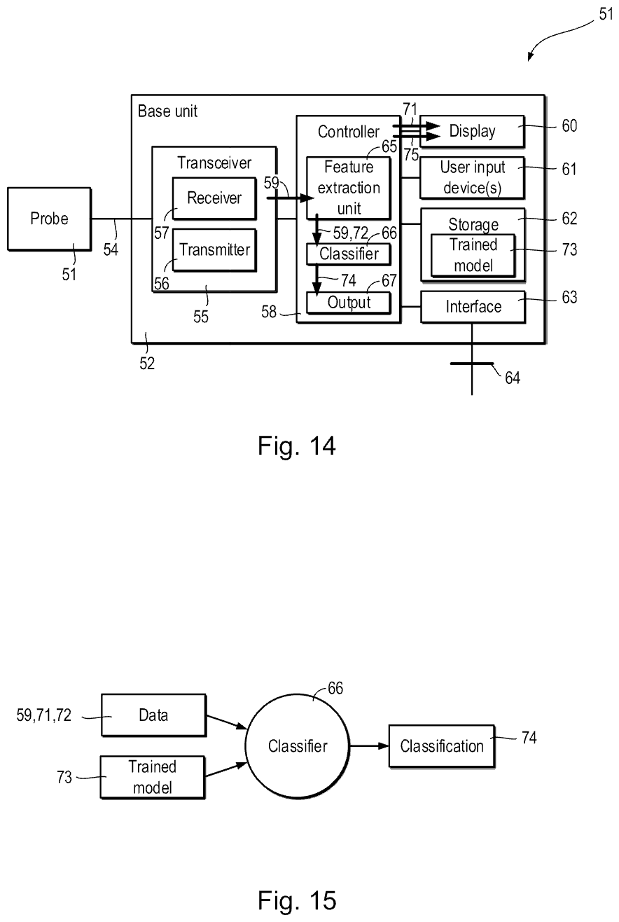

Referring to FIGS. 14 and 15, a medical ultrasound scanner 51 includes a probe 52 containing a transducer array (not shown) and a base unit 53. The probe 52 and base unit 53 may be connected by a wired or wireless link 54. The wireless link 54 may take the form of a BlueTooth® link.

The scanner 51 includes a transceiver 55 which includes a transmitter 56 for generating excitation signals for the transducers (not shown) and a receiver 57. The transceiver 55 is controlled by a controller 58. The controller 58 also processes signals 59 received from the transceiver 55. The base unit 53 includes a display 60, user input devices 61, storage 62 and a wireless interface 63 for communicating via a communications network 64. The transceiver 55 and the controller 58 are housed in the base unit 52. However, in some embodiments the transceiver 55 may be housed in the probe unit 51 and two controllers may be used. A first controller (not shown) may be housed in the probe unit 51 and controls signal excitation and reception. A second controller (not shown) may be housed in the base unit 52 and can be used for signal processing and controlling the user interface. The base unit 52 may take the form of a tablet computer.

The controller 58 implements a feature extraction unit 65, a classifier 66 and classification output unit 67. These may be implemented in software, i.e., using a processor (not shown) in the controller 58 runs code (not shown) stored in memory (not shown) implementing the feature extraction unit 65, classifier 66 and classification output unit 67. However, one or more of the units 65, 66, 67 may be implemented in hardware, for example, using hardware accelerator(s).

The medical ultrasound scanner 51 may be used for conventional Doppler ultrasound and an image 71 may be displayed on the display 60. However, the apparatus 51 also extracts features 72 (such as statistical and/or time-frequency features) from the image 71 or from the signals 59 from the transceiver 55 and supplies the features 72 to the classifier 66 for classification using a trained machine learning model 73. The classifier 66 outputs a classification 74, e.g., PAD or no PAD and, optionally a severity. The classification is passed to the output unit 67 which provides an indication 75 to the operator (not shown) of the classification, for example, by displaying the classification (for example, “PAD” or “No PAD”) on the display 60.

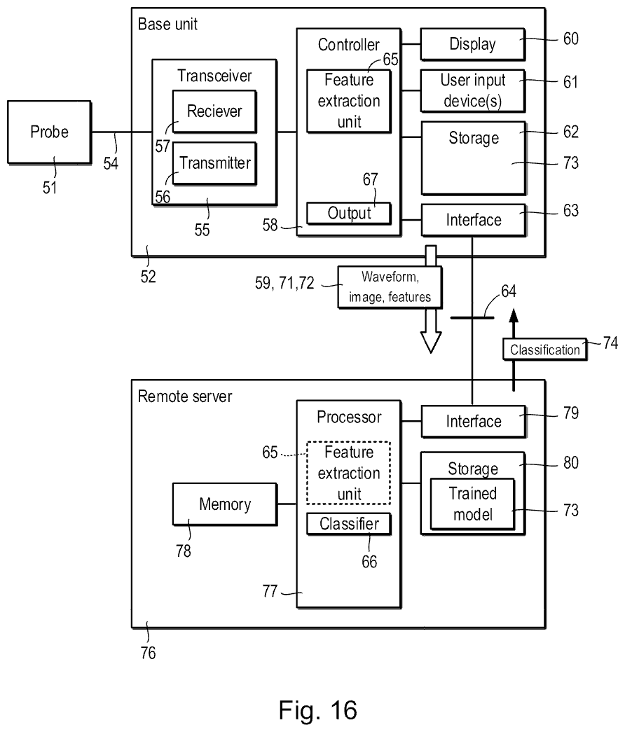

Classification need not be performed locally in the medical ultrasound scanner 51. Instead, classification and, optionally, feature extraction, can be performed remotely in in a remote server 76 (FIG. 16).

Referring to FIG. 16, a remote server 76 includes a processor 77, memory 78, a network interface 79 and storage 80. The server 76 may include hardware accelerator(s).

The medical ultrasound scanner 51 transmits the signal 59, the image 71 and/or features 72 to the server 76 via the communications network 64. The server 76 may, if features 72 have not already been extract them using a feature extraction unit 65. The classifier 66 outputs a classification 74, e.g., PAD or no PAD and, optionally a severity, and the server 74 transmits the classification 74 back to the ultrasonic diagnostic apparatus 51 for the output unit 67 to provide the indication 75 to the operator (not shown).

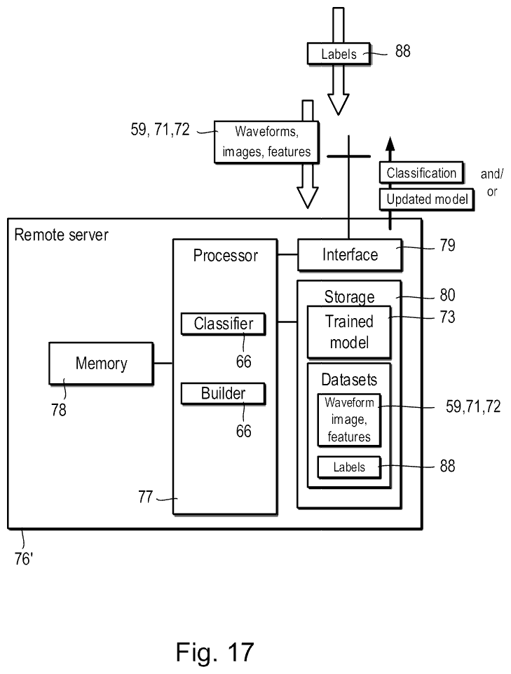

Referring to FIG. 17, a modified server 76′ is shown.

As explained hereinbefore, a large repository of waveforms can be used to increase the size of the dataset available for training. Thus, provided suitable labels 88 are provided, the server 76 can include a builder 66 and can be used to produce an updated trained model 73. For example, data can be 59, 71, 72 can uploaded and stored at the remote server 76′ and an expert (not shown) may inspect the dataset and label the data (e.g., PAD and no-PAD) and update the trained model 73 or generate a new trained model 73. With a larger training set, accuracy, sensitivity and/or specificity can be improved.

Modifications

It will be appreciated that various modifications may be made to the embodiments hereinbefore described. Such modifications may involve equivalent and other features which are already known in the design, manufacture and use Doppler ultrasound systems and component parts thereof and which may be used instead of or in addition to features already described herein. Features of one embodiment may be replaced or supplemented by features of another embodiment.

Pulsed-wave or continuous-wave Doppler ultrasound may be used.

Other features may be used. For instance, other time-frequency features can be extracted, for example, using short-time Fourier transform. Basic spectral analysis features, can be used, for example, power spectral density estimates using fast Fourier transform. Morphological features can be used, such as area under the curve or time for forward flow and/or reverse flow for each waveform complex, systolic rise time (i.e., the time from start of waveform to peak systolic velocity).

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A computer-implemented method, comprising:
classifying an arterial Doppler ultrasound waveform using the arterial Doppler ultrasound waveform and/or a set of features extracted from the arterial Doppler ultrasound waveform using one or more trained machine learning models to identify whether a peripheral arterial disease condition is present and/or to predict a medical outcome related to peripheral arterial disease; and
upon identifying the presence of the peripheral arterial disease condition and/or predicting the medical outcome, signalling the presence of the peripheral arterial disease condition and/or the medical outcome.

2. The method of claim 1, further comprising:
receiving arterial Doppler ultrasound waveform; and
extracting the features from arterial Doppler ultrasound waveform.

3. The method of claim 1, wherein the features include:
a set of time-domain statistical features; and/or
a set of time-frequency domain features.

4. The method of claim 3, wherein the set of time-domain statistical features includes at least one selected from the group consisting of:
kurtosis,
skewness,
peak value,
mean,
standard deviation, STD,
root mean square, RMS,
impulse factor,
crest factor,
clearance factor,
signal to noise ratio, SNR,
total harmonic distortion, THD,
signal to noise and distortion ratio, SINAD, and
shape factor.

5. The method of claim 4, wherein the set of time-domain statistical features includes all of the features in the group.

6. The method of claim 1, further comprising:
receiving an image of the arterial Doppler ultrasound waveform; and
reconstructing the arterial Doppler ultrasound waveform from the image.

7. The method of claim 1, further comprising:
performing signal smoothing of the arterial Doppler ultrasound waveform prior to extracting features.

8. The method of claim 1, wherein classifying the arterial Doppler ultrasound waveform using the arterial Doppler ultrasound waveform using one or more trained machine learning models comprises:
using a first machine learning model which is a recurrent neural network.

9. The method of claim 8, wherein the recurrent neural network is a long short-term memory network.

10. The method of claim 1, wherein classifying the features extracted from the arterial Doppler ultrasound waveform using one or more trained machine learning models comprising:
using a second machine learning model which is based on a supervised machined learning algorithm.

11. The method of claim 10, wherein the second machine learning model is based on a support-vector machine or logistic regression.

12. (canceled)

13. A computer program product comprising a computer-readable medium which is non-transitory storing thereon a computer program which, when executed by at least one processor, performs the method of claim 1.

14. A machine learning classifier, comprising:
at least one processor;
the at least one processor configured:
to classify an arterial Doppler ultrasound waveform using the arterial Doppler ultrasound waveform and/or a set of features extracted from the arterial Doppler ultrasound waveform using one or more trained machine learning models to identify whether a peripheral arterial disease condition is present and/or to predict a medical outcome related to peripheral arterial disease; and
upon identifying the presence of the peripheral arterial disease condition and/or predicting the medical outcome, to signal the presence of the peripheral arterial disease condition and/or the medical outcome.

15. The machine learning classifier of claim 14, wherein the at least one processor is configured:
to receive the arterial Doppler ultrasound waveform; and
to extract the features from the arterial Doppler ultrasound waveform.

16. A medical ultrasound scanner comprising:
an ultrasound transceiver for generating an arterial Doppler ultrasound waveform;
an optional signal processor; and
the machine learning classifier of claim 14;
wherein:
the ultrasound transceiver is configured to provide the arterial Doppler ultrasound waveform to the signal processor and/or to the system; and
the signal processor is configured to extract features from the arterial Doppler ultrasound waveform and to provide the features to the system.

17. A system comprising:
a medical ultrasound scanner having a communications network interface; and
a server having a communications interface, the server comprising the machine learning classifier of claim 14;
wherein:
the medical ultrasound scanner is configured to transmit the arterial Doppler ultrasound waveform and/or a set of features to the server; and
the server is configured to identify the presence of the peripheral arterial disease condition and/or the medical outcome to the medical ultrasound scanner or another device.

18. A computer-implemented method, comprising:
training one or more machine learning trainers using a plurality of arterial Doppler ultrasound waveforms as a training set and/or a plurality of sets of features extracted from respective arterial Doppler ultrasound waveforms as a training set, wherein each one of the plurality of arterial Doppler ultrasound waveforms and each one of the sets of features are labelled as to the presence of a peripheral arterial disease condition and/or a prediction of a medical outcome related to peripheral arterial disease; and
storing one or more trained machine learning models obtained from training the one or more machine learning trainers.

19. The method of claim 18, wherein the one or more machine learning trainers includes:
a first machine learning trainer is based on a recurrent neural network; and/or
a second machine learning trainer is based on a support-vector machine or logistic regression.

20. A machine learning trainer, comprising:
at least one processor; and
storage;
the at least one processor configured:
to train one or more machine learning trainers using a plurality of arterial Doppler ultrasound waveforms as a training set and/or a plurality of sets of features extracted from respective arterial Doppler ultrasound waveforms as a training set, wherein each one of the plurality of arterial Doppler ultrasound waveforms and each one of the sets of features are labelled as to the presence of a peripheral arterial disease condition and/or a prediction of a medical outcome related to peripheral arterial disease; and
to store one or more trained machine learning models obtained from training the one or more machine learning trainers.
